(19) United States
(12) Patent Application Publication
(10) Pub. No.: US 2008/0241848 A1
(43) Pub. Date: Oct. 2, 2008

(54) METHODS FOR PRENATAL DIAGNOSIS OF ANEUPLOIDY

(75) Inventors: Petros Tsipouras, Madison, CT (US); Triantafyllos Tafas, Rocky Hill, CT (US)

Correspondence Address: KELLEY DRYE & WARREN LLP, 400 ALTLANTIC STREET , 13TH FLOOR, STAMFORD, CT, 06901, US

(73) Assignee: IKONISYS, INC., NEW HAVEN, CT (US)

(21) Appl. No.: 12/117,281

(22) Filed: May 8, 2008

Related U.S. Application Data
(63) Continuation-in-part of application No. 10/091,360, filed on Mar. 4, 2002.
Application No. 10/091,360 is a continuation of application No. 09/724,384, filed on Nov. 28, 2000, now abandoned.
Application No. 09/724,384 is a division of application No. 09/421,956, filed on Oct. 20, 1999, now abandoned.
Application No. 09/421,956 is a continuation of application No. PCT/US99/10026, filed on May 7, 1999.
(60) Provisional application No. 60/084,893, filed on May 9, 1998.

(51) Int. Cl. C12Q 1/68

(52) U.S. Cl. 435/6

(57) ABSTRACT

Methods are disclosed for the automated prenatal genetic diagnosis of aneuploidy using an automated fluorescence microscope, conducted on samples of maternal blood that have been hybridized with FISH probes.

### CROSS-REFERENCE TO RELATED APPLICATIONS

This Utility Patent Application is a Continuation-In-Part (CIP) Application of co-pending U.S. patent application Ser. No. 10/091,360, published as U.S. Patent Application Publication No. 2002/0160443, which is a continuation of U.S. patent application Ser. No. 09/724,384 filed Nov. 28, 2000, which is a divisional of U.S. application Ser. No. 09/421,956 filed Oct. 20, 1999, which is a continuation of PCT/US99/10026 filed May 7, 1999, which claims priority of U.S. Provisional Patent Application No. 60/084,893, filed May 9, 1998, which is incorporated by reference herein in its entirety

All references cited in this specification, and their references, are incorporated by reference herein where appropriate for teachings of additional or alternative details, features, and/or technical background.

### FIELD OF THE INVENTION

The present invention relates to computer controlled methods and apparatus for obtaining and preparing cell samples and for identifying a rare cell of interest from a field of cells and making a diagnosis based on a characteristic of a rare cell selected in the field. In one important embodiment, the invention relates to obtaining and preparing a maternal blood sample for fetal cell based prenatal diagnosis.

### BACKGROUND OF THE INVENTION

The advent of DNA based prenatal diagnosis for human genetic disorders has led to the development of a number of new diagnostic methods. These diagnostic methods permit early detection and consequently informed decisions and intervention with respect to fetus having a genetic disorder. These methods, however, have a number of disadvantages. Each of the new diagnostic methods with which this discussion is concerned requires that a sample of isolated fetal cells be obtained, so that the DNA of the fetus may be examined or tested for signs of specific genetic disorders. The disadvantages of these modern methods stem primarily from the need to obtain a sample of fetal cells. Currently, fetal cells are obtained by invasive procedures requiring obstetric intervention by amniocentesis or by chorionic villus sampling. These highly specialized procedures carry a small, but significant, risk to the fetus. Early in pregnancy, the level of risk to the fetus is high and the number of cells obtained is low. Therefore, results of these procedures often are not obtained until 18-20 weeks of pregnancy.

One modern procedure for obtaining fetal cells relies on leakage of fetal cells into the maternal circulation. By simply drawing a sample of maternal blood, it is theoretically possible to obtain fetal cell material in a sufficient quantity for prenatal diagnosis by DNA based methods. Obtaining fetal cells from the maternal blood circulation avoids any risk to the fetus and can be undertaken as early as 10-12 weeks of pregnancy.

Fetal cells which have been detected in the maternal blood circulation include trophoblasts, lymphocytes and nucleated erythrocytes. Trophoblasts were the first fetal cells to be identified in the maternal blood circulation, due to their large size. However, nucleated erythrocytes have generated the greatest degree of interest as sources of genetic material for prenatal diagnosis due to their rarity in the adult blood circulation, their abundance in fetal blood and their limited life span. These factors combine to reduce errors in distinguishing fetal cell material from maternal cell material. Fetal cells circulating in the maternal blood have a life span ranging from a few weeks (for the nucleated erythrocytes) to a few years (for the lymphocytes).

Although they are consistently present in the maternal blood circulation, fetal cells are very rare, severely limiting their diagnostic utility. Estimates of the concentration of fetal cells within the maternal blood circulation vary widely, from a high level of 1 fetal cell in 10.sup.5 maternal cells, to a low level of one fetal cell in 10.sup.9 maternal cells. Thus, a 10 ml sample of maternal blood will ordinarily contain between about 10 and 100 fetal cells. Throughout this description, the concentration of fetal cells found in a freshly drawn maternal blood sample, prior to any further treatment, is referred to as the “naturally present concentration” of fetal cells, typically, but not necessarily, within the above ranges. Also throughout this description, the term “unenriched maternal blood” shall refer to a sample of maternal blood which contains only a naturally present concentration of fetal cells.

Since the naturally present concentration of fetal cells in unenriched maternal blood is so low, in order to obtain a diagnostically significant sample of fetal cells modern techniques include methods of physically isolating the fetal cells from the maternal cells in the sample. In essence, modern techniques are methods of concentrating the fetal cells within a sample, i.e., enriching the sample, for example by removing excess maternal cells, without removing fetal cells. These methods are extremely difficult to perform, often fail to isolate a sufficient number of fetal cells to be diagnostically significant and sometimes fail to provide a sample of a sufficient number of undamaged fetal cells of adequate purity for reliable subsequent diagnosis.

The placenta does not constitute an absolute barrier against the translocation of cells from a fetus to the maternal circulation. It has become apparent over the past 10-15 years that fetal cells can traverse the placenta and enter the circulation of the mother. This offers the potential for developing diagnostic procedures addressed to the fetus simply by using a blood sample drawn from the mother. This avoids more complex and risk-prone procedures such as amniocentesis.

Several congenital defects are due to aneuploidy of a particular chromosome arising in an embryo or fetus. Such a defect can be a monogenic disorder (such as an autosomal recessive, an autosomal dominant or an X-linked disorder), or a chromosomal structural aberration (such as a balanced translocation). Nonlimiting examples of monogenic disorders include cystic fibrosis, beta thalassemia, sickle cell disease, spinal muscular atrophy type 1 myotonic dystrophy, Huntington's disease, and Charcot-Marie-Tooth disease. X-linked diseases include fragile X syndrome, hemophilia A, and Duchenne muscular dystrophy. Additionally, chromosomal abnormalities such as reciprocal and Robertsonian translocations, and other abnormalities such as chromosomal inversions or deletions can be detected. Other congenital diseases include Down Syndrome (Trisomy 21), and Tay Sachs disease. Monosomy in humans includes Turner syndrome (X0, in which only one X chromosome instead of the usual two found in a normal female); cri du chat syndrome (a partial monosomy caused by a deletion of the end of the 5p chromosome), and 1p36 Deletion Syndrome—a partial monosomy.

Trisomy in humans commonly results in spontaneous abortion; the most common types that survive to birth in humans are trisomy 21 (Down syndrome; the most common trisomy in viable births), trisomy 18 (Edwards syndrome), trisomy 13 (Patau syndrome), trisomy 12 (a prognostic indicator of chronic lymphocytic leukemia), trisomy 9, trisomy 8 (Warkany syndrome 2), and trisomy 16 (the most common trisomy in humans, occurring in more than 1% of pregnancies, usually resulting in spontaneous miscarriage).

Fluorescence microscopy of cells and tissues, involving treating cells with fluorescent reagents and imaging the cells, is well known in the art. These methods have been designed and optimized for high spatial and temporal resolution in measurements of the distribution, amount and biochemical environment of the fluorescent reporter molecules in cells. Detection of fluorescent signals may be by way of an epifluorescent microscope which uses emitted fluorescent light to form an image (whereas a conventional reflecting microscope uses scattered illumination light to form an image). An advantage of an epifluorescence microscope is that the sample may be prepared such that the fluorescent molecules are preferentially attached to the biological structures of interest thereby allowing identification of such biological structures of interest.

Automated methods of conducting microscopic analysis of biological samples enhance diagnostic procedures and optimize the throughput of samples in a microscope-based diagnostic facility. Various co-owned U.S. patent applications, described more fully below, disclose aspects and embodiments of apparatuses and methods for automated microscopic analysis. These include an integrated robotic microscope system, a dynamic automated microscope operation and slide scanning system, various interchangeable objective lenses, filters, and similar elements for use in an automated microscope system, an automated microscope stage for use in an automated microscope system, an automated microscope slide cassette and slide handling system for use in an automated microscope system, an automated microscope slide loading and unloading mechanism for use in an automated microscope system, automated methods that employ computer-resident programs to drive the microscopic detection of fluorescent signals from a biological sample, useable to drive an automated microscope system, automatic operation of a microscope using computer-resident programs to drive the microscope in conducting a FISH assay for image processing.

A method of scanning and analysis of cytology and histology samples using a flatbed scanner to capture images of the structures of interest for the analysis of common pathology staining techniques is disclosed in U.S. Pat. No. 7,133,543 issued Nov. 7, 2006.

The fluorescent dye 4′,6-diamidino-2-phenylindole (DAPI; CAS number: [28718-90-3]) binds strongly to DNA. It is used extensively in fluorescence microscopy Since DAPI will pass through an intact cell membrane, it may be used to stain live and fixed cells. DAPI is excited with ultraviolet light. When bound to double-stranded DNA its absorption maximum may be about 358 nm and its emission maximum may be about 461 nm, (a blue color). DAPI will also bind to RNA, though it is not as strongly fluorescent. Its emission shifts to about 400 nm when bound to RNA. DAPI's blue emission is convenient for microscopists who wish to use multiple fluorescent stains in a single sample. There is very little fluorescence overlap, for example, between DAPI and green-fluorescent molecules like fluorescein and green fluorescent protein (GFP), or red-fluorescent dyes like Texas Red. Other fluorescent dyes are used to detect other biological structures.

Fluorescence in situ hybridization (FISH) is commonly used for the detection of chromosomal abnormalities (for instance, aneuploidy screening or chromosomal translocations). FISH uses fluorescent polynucleotide probes which hybridize only to those parts of the chromosome with which they show a high degree of sequence similarity. Such tags are directed to specific chromosomes and specific chromosome regions. The probe must hybridize specifically to its target (and not to similar sequences in the genome) but not to unintended loci, and it should be tagged directly with fluorophores. This can be done in various ways, for example nick translation and PCR using tagged nucleotides. If signal amplification is necessary to exceed the detection threshold of the microscope (which depends on many factors such as probe labelling efficiency, the kind of probe and the fluorescent dye), fluorescent tagged antibodies or streptavidin are bound to the tag molecules, thus amplifying the fluorescence.

Currently, many probes are available for different segments of all chromosomes, but the limited number of different fluorochromes confines the number of signals that can be analysed simultaneously. The type and number of probes that are used on a sample depends on the indication. The use of probes for chromosomes X, Y, 13, 14, 15, 16, 18, 21 and 22 has the potential of detecting 70% of the aneuploidies found in spontaneous abortions. In the case of chromosome rearrangements, specific combinations of probes have to be chosen that flank the region of interest. The FISH technique is considered to have an error rate between 5 and 10%.

Fetal cells have been found to traverse the placental barrier and enter the maternal circulation. For example, it has been found that red cells of fetal origin can be as high as 5% of the total cells in a matemal blood sample (Porra, V et al., Transfusion, 2007 47(7):1281-9). Purwosunu, Y et al. (Taiwan J. Obstet. Gynecol. 2006 45(1):10-20) note the prevalence of nucleated fetal erythrocytes in maternal circulation, and review the difficulty of separating these cells from maternal cells for assay. Shaffer, L G, et al. (Am. J. Med. Gen. C Semin. Med. Genet. 2007 145(1):87-98) review the use of various methods including FISH in fetal cytogenetic analysis.

In addition, free fetal DNA has been found circulating in maternal blood. Montagnana, M et al. (Minerva Ginecol. 2007 59(3):331-341) suggest its use in various fetal diagnostic assays. Fetal free DNA is only a small fraction of the total free DNA circulating in maternal plasma (Li, Y, et al., Ann. NY Acad. Sci. 2006 1075:81-7). Selectivity of genetic analysis is thereby rendered difficult. In assessing the quantitation of fetal cells in maternal circulation Zhong, X Y et al. (Prenat. Diagn. 2006 26(9):850:4) report about 2 cells per mL of maternal blood, and note that in various circumstances free fetal DNA can complicate or interfere with the quantitation process.

The present state of the field of prenatal genetic diagnosis reveals a strong need for a diagnostic procedure that is free of significant risk to both the mother and the fetus, is highly sensitive, and accurate. Further there is a need for a method of prenatal fetal genetic analysis that is capable of operating in a high background of material of maternal origin. In addition there remains a need for a procedure susceptible of automation. Such methods should be well suited to provide true positive determinations, which are likely to occur in only small numbers against a background of large numbers of cells yielding a negative result.

### SUMMARY OF THE INVENTION

It is desired to provide a computer controlled method and apparatus for detecting and diagnosing a rare cell type in a tissue sample, said diagnosis being based upon a characteristic of that rare cell. It is further desired to provide a computer controlled method and apparatus for detecting fetal cells in a blood preparation and performing a fetal cell based prenatal diagnosis that solves the above-identified problems, which overcomes such other problems and meets such other goals as will be apparent to the person skilled in this art after reading a description of the invention.

Generally, the invention provides a computer-implemented method of processing body fluid or tissue sample image data, the method comprising creating a subset of a first image data set representing an image of a body fluid or tissue sample taken at a first magnification, the subset representing a candidate blob which may contain a rare cell creating a subset of a second image data set representing an image of the candidate blob taken at a second magnification, the subset of the second data set representing the rare cell and storing the subset of the second data set in a computer memory.

In general, a subset of a first image data set can be created by observing an optical field of a monolayer of cells from a body fluid or tissue sample using a computerized microscopic vision system to detect a signal indicative of the presence of a rare cell.

The method further comprises contacting a body fluid or tissue sample at a location corresponding to each candidate blob represented in the subset of the first image data set, with a reagent to generate a medically significant signal. This method provides the advantage of being able to remove from further processing a body fluid or tissue sample for which no subset of the first data set representing a candidate blob is created. The signal can be measured to determine whether it is a significant signal level. The first and/or the second image data subsets can be transformed into a representation that is more suitable for control and processing by a computer as described herein. In a preferred embodiment, the image data is transformed from an RGB (Red Green Blue) signal into an HLS (Hue Luminescence Saturation) signal. Filters and/or masks are utilized to distinguish those cells that meet pre-selected criteria and eliminate those that do not, and thus identify rare cells.

In certain embodiments a method of prenatal genetic diagnosis of aneuploidy is provided. First, a specimen of blood from a pregnant human female is obtained. Next, at least a portion of the specimen is exposed to conditions that lyse cells contained in the blood specimen, thus providing a complex mixture of components including cell nuclei, and the lysed specimen is deposited on a microscope slide. Then the deposited lysed specimen is contacted with at least one FISH probe specific for a chromosome known to exhibit aneuploidy, under experimental conditions that promote hybridization of the probe to a target nucleic acid sequence comprised in the nuclei. In various additional embodiments the lysed specimen is subjected to conditions that fix components within the nuclei prior to placement on a slide; whereas in alternative embodiments the lysed specimen deposited on the slide is subjected to conditions that fix components within the nuclei. Next, a fluorescent microscopic image of the specimen with any fluorescent probe bound is obtained using an automated fluorescence microscope system, such that the image represents a chromosome having a FISH probe hybridized to it. The image is then automatically analyzed for aneuploidy, and the results of the analysis are automatically reported. In various embodiments the analysis in conducted automatically using software resident in the automated microscope system. The automated steps involving the microscope and computer resident analysis and reporting, are carried out in the absence of intervention by a human. In additional embodiments more than one FISH probe specific for the potentially aneuploid chromosome may be used. The ;plurality of probes may include the identical fluorescent label, or different labels may be used in different probes. In various particular embodiments, the chromosome targeted by the FISH probe is chromosome 21, when it is intended to analyze for Down syndrome. In various other embodiments, the targeted chromosome may be chromosome 13, 18 or 16.

In additional embodiments a method for high throughput prenatal genetic diagnosis of aneuploidy is provided. In these embodiments, first, at least one specimen on a microscope slide is provided, wherein the specimen includes nuclei derived from a sample of blood from a pregnant human female that have been hybridized to at least one FISH probe specific for a chromosome known to exhibit aneuploidy. In various additional embodiments more than one FISH probe specific for the potentially aneuploid chromosome may be used. The plurality of probes may include the identical fluorescent label, or different labels may be used in different probes. Next, at least one specimen-bearing slide is installed in a means for automated, reversible, placement of the slide on the stage of an automated fluorescence microscope. In various embodiments the means includes a slide-holding cassette, and the cassette may be controlled for automated operation. Next, the slide is caused to be reversibly placed on the microscope stage. This process is reversible such that a given slide may be withdrawn under automated operation, after an image is captured, and a new slide placed on the stage. Next, the microscope is caused automatically to obtain at least one image of the specimen including a locus on the slide comprising a fluorescent probe bound to a chromosome. In various embodiments automated operation of the microscope seeks out a plurality of loci within the specimen whose image contains a fluorescent probe bound to a chromosome. Next, the image is analyzed in an automated fashion in order to assess the state of ploidy of a chromosome at the locus, and the results of the analysis are automatically reported. The steps of placing a slide on the stage, obtaining an image thereof, analyzing the image and reporting the results are repeated for a plurality of slides that have been placed in the means for placement of the slide on the microscope stage. In addition the steps involving automated operation of the microscope and the computer resident analysis and reporting functions are carried out intervention by a human. In various particular embodiments, the chromosome targeted by the FISH probe is chromosome 21, when it is intended to analyze for Down syndrome. In various other embodiments, the targeted chromosome may be chromosome 13, 18 or 16.

### BRIEF DESCRIPTIONS OF DRAWINGS

In the accompanying drawings, in which like reference designations indicate like elements:

FIG. 1 is a flow chart summarizing the method of one aspect of the invention;

FIG. 2 is a block diagram of an analysis system used in one embodiment of one aspect of the invention;

FIG. 3 is a flow chart of stage I leading to detecting the first signal; F

FIGS. 4A and 4B taken together are a flow chart of stage 1I leading to detecting the first signal;

FIG. 5 is a flow chart of detection of the second signal;

FIG. 6 is a schematic representation of a variation of an apparatus embodying aspects of the invention, using a continuous smear technique;

FIG. 7 is a block diagram of an analysis and reagent dispensing system used in one embodiment of one aspect of the invention;.

FIG. 8 is an outline of a multiple objective microscopy system; and

FIG. 9 is an image “composition” method.

### DETAILED DESCRIPTION OF THE INVENTION

The invention will be better understood upon reading the following detailed description of the invention and of various exemplary embodiments of the invention, in connection with the accompanying drawings. While the detailed description explains the invention with respect to fetal cells as the rare cell type and blood as the body fluid or tissue sample, it will be clear to those skilled in the art that the invention can be applied to and, in fact, encompasses diagnosis based on any rare cell type and any body fluid or tissue sample for which it is possible to create a monolayer of cells on a substrate.

Body fluids and tissue samples that fall within the scope of the invention include but are not limited to blood, tissue biopsies, spinal fluid, meningeal fluid, urine, alveolar fluid, etc. For those tissue samples in which the cells do not naturally exist in a monolayer, the cells can be dissociated by standard techniques known to those skilled in the art. These techniques include but are not limited to trypsin, collagenase or dispase treatment of the tissue.

In an important specific embodiment, the invention is used to detect and diagnose fetal cells. Our approach is directly opposite to that taken by others seeking a non-invasive method for performing fetal cell based prenatal diagnosis. Rather than attempting substantially to enrich the concentration of fetal cells within a maternal blood sample, our approach involves identifying fetal cells within an unenriched maternal blood sample and subsequently performing diagnostic procedures on the fetal cells so identified, in situ.

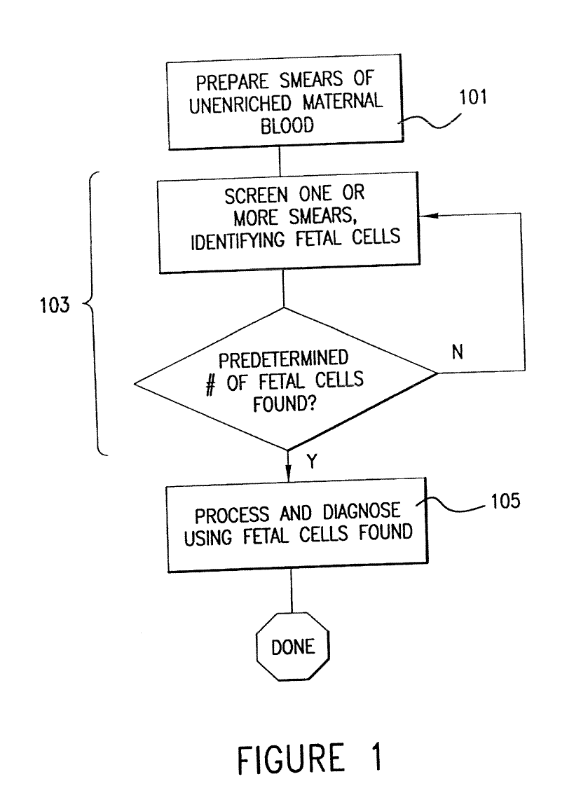

A summary of this new approach, shown in the flow chart of FIG. 1, is as follows:

Prepare one or more blood smears from a sample of unenriched maternal blood 101;

Screen the one or more blood smears until a predetermined number of fetal cells (e.g., nucleated erythrocytes) have been identified and their coordinates defined 103; and

Process those smears or coordinates of a smear at which fetal cells have been identified, diagnosing the presence or absence of a particular genetic feature in the fetal cells 105.

In this approach, two signals are defined, referred to hereinafter as the first signal and the second signal. As used herein, “signal” should be taken in its broadest sense, as a physical manifestation which can be detected and identified, thus carrying information. One simple and useful signal is the light emitted by a fluorescent dye selectively bound to a structure of interest. That signal indicates the presence of the structure, which might be difficult to detect absent the fluorescent dye.

Screening 103 is based on the first signal. The first signal, which in this exemplary embodiment indicates cell identity, may be generated by a fluorescent dye bound to an antibody against the hemoglobin .epsilon.-chain, i.e., embryonal hemoglobin, for example.

Alternatively, for example, a metric of each cell's similarity to the characteristic morphology of nucleated erythrocytes, discerned using cell recognition algorithms may serve as the first signal. In yet another example, the first signal may be a measure of the presence of the characteristic color of fetal hemoglobin after staining with eosin and acid hematoxylin. It should now be evident that any detectable indicator of the presence of fetal cells may serve as the first signal, subject to certain constraints noted below.

Diagnosing 105 is based on the second signal. The second signal, which in this exemplary embodiment indicates the presence of a particular genetic characteristic being tested for, may be generated, for example, by in situ PCR-amplification or PCR in situ hybridization or FISH. Cells that emit both signals, i.e., the cell is a fetal cell and contains the genetic characteristic being tested for, will be scored. Counts may be maintained of the number and strengths of the first and second signals detected.

In one embodiment, a specific nucleic acid sequence is detected by FISH. In an exemplary embodiment, FISH comprises hybridizing the denatured test DNA of the rare cell type, e.g. a fetal cell, with a denatured dioxygenin (DIG)-labeled genomic probe. The samples containing the test DNA are washed and allowed to bind to an anti-DIG antibody coupled to a fluorophore. Optionally, a second layer of fluorophore (e.g. FITC) is added by incubation with fluorophore-conjugated anti-Fab antibodies. In a preferred embodiment, FISH comprises hybridizing the denatured DNA of the rare cell with a fluorescently labeled probe comprising DNA sequence(s) homologous to a specific target DNA region(s) directly labeled with a particular fluorophore.

Automated sample analysis will be performed by an apparatus and method of distinguishing in an optical field objects of interest from other objects and background. An example of an automated system is disclosed in our U.S. Pat. No. 5,352,613, issued Oct. 4, 1994. Furthermore, once an object has been identified, the color, i.e., the combination of the red, green, blue components for the pixels that comprise the object, or other parameters of interest relative to that object can be measured and stored.

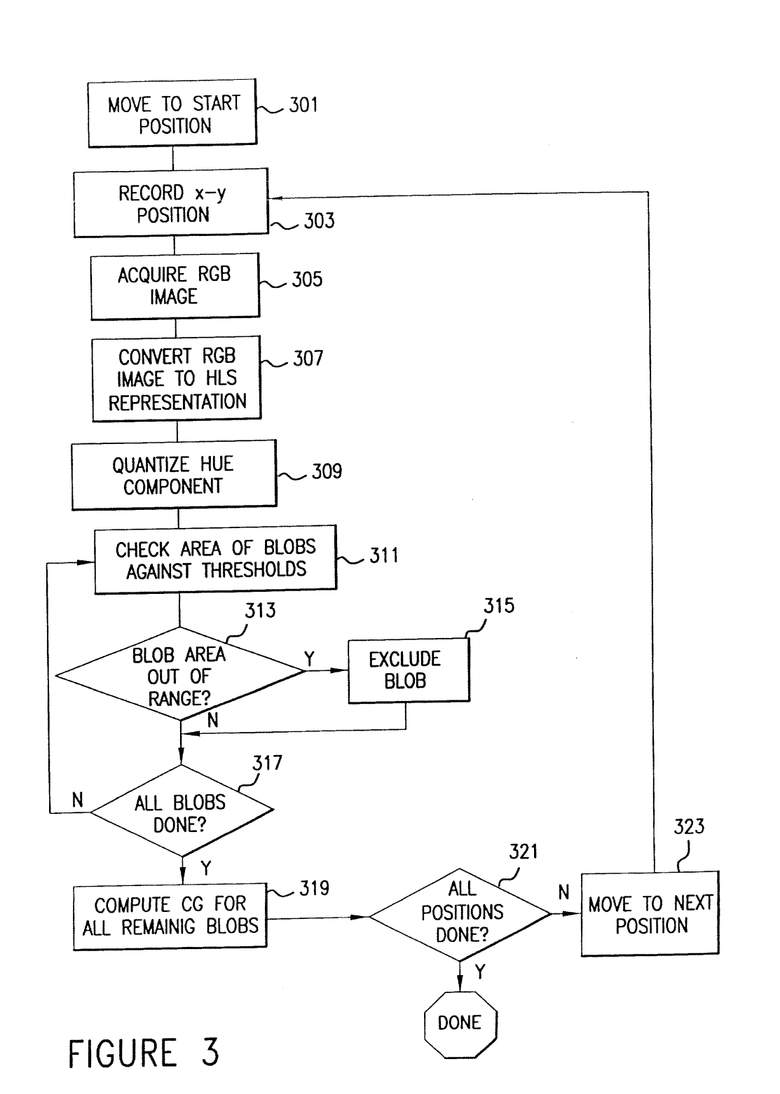

Another example of an apparatus and method for automated sample analysis is presented, infra, in Section 6, Exemplary Embodiments, in particular Sections 6.2.1, 6.2.2 and 6.3, and is illustrated in FIGS. 3-5.

In one embodiment of the invention, the system consists of an automatic microscopical sample inspection system having:

a sample storage and loading and unloading module

a sample transporting mechanism to and from an automated stage that moves the sample under a microscope objective lenses array

an array of CCD cameras

a processing unit having a host computer, multiple controllers to control all mechanical parts of the microscopy system and

a high speed image processing unit where the CCD cameras are connected.

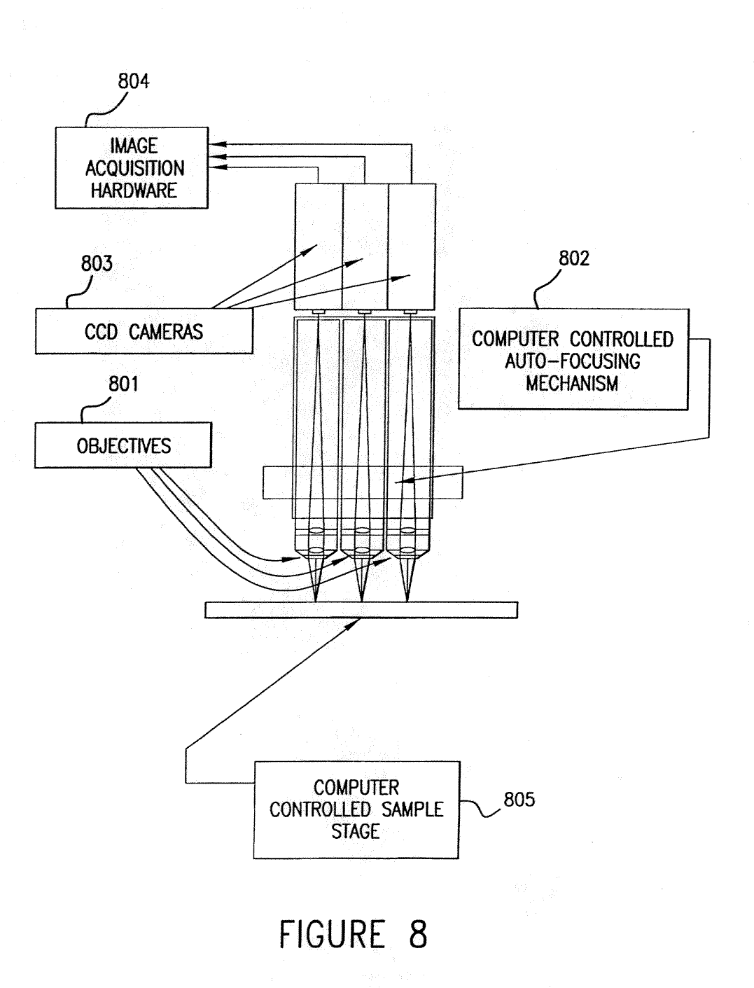

An innovative feature of this embodiment of a computer controlled system is an array of two or more objective lenses having the same optical characteristics, depicted in FIG. 8. The lenses are arranged in a row and each of them has its own z-axis movement mechanism, so that they can be individually focused (801). This system can be equipped with a suitable mechanism so that the multiple objective holder can be exchanged to suit the same variety of magnification needs that a common single-lens microscope can cover. Usually the magnification range of light microscope objectives extends from 1.times. to 100.times.

Each objective is connected to its own CCD camera (803). The camera field of view characteristics are such that it acquires the full area of the optical field as provided by the lens.

Each camera is connected to an image acquisition device (804). This is installed in a host computer. For each optical field acquired, the computer is recording its physical location on the microscopical sample. This is achieved through the use of a computer controlled x-y mechanical stage (805). The image provided by the camera is digitized and stored in the host computer memory. With the current system, each objective lens can simultaneously provide an image to the computer, each of which comprises a certain portion of the sample area. The lenses should be appropriately corrected for chromatic aberrations so that the image has stable qualitative characteristics all along its area.

The imaged areas will be in varying physical distance from each other. This distance is a function of the distance at which the lenses are arranged and depends on the physical dimensions of the lenses. It will also depend on the lenses' characteristics, namely numerical aperture and magnification specifications, which affect the area of the optical field that can be acquired. Therefore, for lenses of varying magnification/numerical aperture, the physical location of the acquired image will also vary.

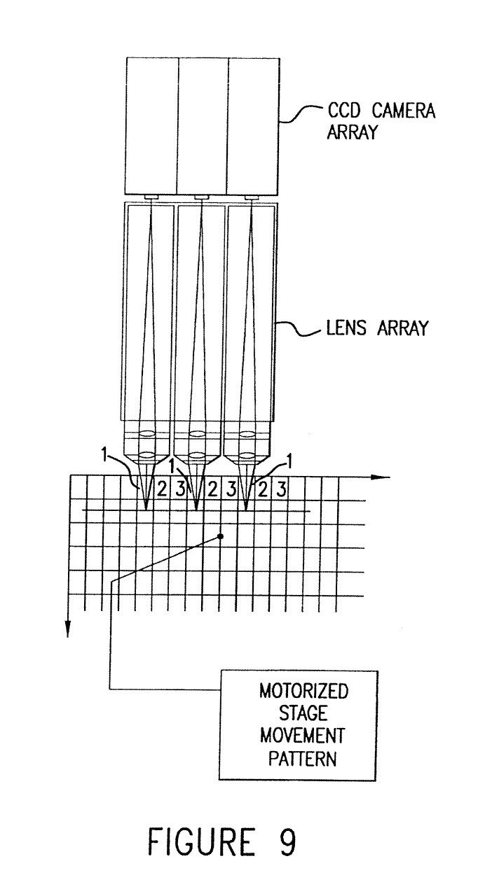

The computer will keep track of the features of the objectives-array in use as well as the position of the motorized stage. The stored characteristics of each image can be used in fitting the image in its correct position in a virtual patchwork, i.e. “composed” image, in the computer memory as shown in FIG. 9.

For example, when starting the host computer moves the sample stage to an initial (x.sub.1,y.sub.1) position. Following the acquisition of the images at this position, the stage moves to a new (x.sub.2,y.sub.2) position, in a side-wise manner. Then a new set of images is acquired and also stored. As shown in FIG. 9 at a certain step 1, the image segments denoted “1” are captured and stored. In step 2, the segments “2” are stored. In step 3, the segments “3” are stored. The complete image is “composed” in the computer memory as the successive image segments are acquired.

The host computer system that is controlling the above configuration, is driven by software system that controls all mechanical components of the system through suitable device drivers. The software also comprises properly designed image composition algorithms that compose the digitized image in the computer memory and supply the composed image for processing to further algorithms. Through image decomposition, synthesis and image processing specific features particular to the specific sample are detected.

In all automated sample analysis embodiments of the invention, if the generation of the first signal is measured first, indicating cell identity, the one or more smears will be observed using an automated optical microscope to delineate coordinates of a desired number of fetal cells. Only those smears found to contain fetal cells need be treated to generate the second signal, indicating the presence of the particular genetic characteristic being tested for. The automated image analysis algorithms will search for the presence of the second signal at predetermined coordinates of fetal cells and also at predetermined coordinates of control maternal cells. This process could be reversed, whereby the genetic abnormality signal is observed first, and then the cell emitting that signal could be observed to determine whether it is a fetal cell. It is even possible to observe both signals simultaneously, searching only for the simultaneous presence of two signals at a single set of coordinates or even a single signal which results from the interaction of two components (e.g. a quenching of a first signal by a partner ‘signal’, the first signal being for the cell type and the partner ‘signal’ being for the genetic abnormality).

The requirements and constraints on the generation of the first and second signals are relatively simple. The materials and techniques used to generate the first signal should not interfere adversely with the materials and techniques used to generate the second signal (to an extent which compromises unacceptably the diagnosis), and visa versa. Nor should they damage or alter the cell characteristics sought to be measured to an extent that compromises unacceptably the diagnosis. Finally, any other desirable or required treatment of the cells should also not interfere with the materials or techniques used to generate the first and second signals to an extent that compromises unacceptably the diagnosis. Within those limits, any suitable generators of the first and second signals may be used.

This exemplary embodiment of the invention may be characterized thus: (i) rather than attempting to enrich (or to further enrich if already partially enriched) the concentration of fetal cells within the maternal blood, fetal cells within the unenriched maternal blood sample are identified for further processing; and (ii) a suitable single cell detection method, such as in situ PCR and/or PCR in situ hybridization is performed to determine the presence of a genetic characteristic being tested for, in some instances only on smears or coordinates of smears that have already been stained and processed, and within which fetal cells have been detected.

Although in an important embodiment, the maternal blood used contains a naturally present concentration of fetal cells, the invention is meant to embrace also maternal blood which has been partially enriched for fetal cells. According to the prior art, the goal was to obtain as much enrichment as possible, to achieve concentrations of fetal cells greater than one fetal cell per 1000 maternal cells. It in particular was the goal to completely isolate fetal cells from maternal cells. According to the invention, cell samples are used where the rare cell is present at no greater than one in every 10,000 cells (i.e. no greater than 0.01%). Thus, simple procedures may be employed to partially enrich the maternal blood sample for fetal cells, such as using simple fractionation procedures (e.g. centrifugation or density gradients) and the like. The procedure falls within the scope of the invention when the sample of cells containing the rare cell is used where the rare cell is present at no greater a concentration than 0.01%. As mentioned above, the invention also in very important embodiments is used where the concentration of the rare cell is 0.001%, 0.0001%, 0.00001%, 0.000001%, and even 0.0000001%. The typical concentration of fetal cells in maternal blood is between one fetal cell in 10.sup.5 maternal cells to one fetal cell in 10.sup.9 maternal cells. Thus, the invention is useful over the full-range of concentrations of fetal cells in maternal blood as typically occurs naturally.

In one specific embodiment of the invention, when the rare cell type is present in the sample, the method of the invention detects the rare cell type at a frequency of no less than 80%. In other embodiments, the detection frequencies are no less than 85%, 90%, 95% and 99%.

In addition to the detection of genetic abnormalities in a developing fetus, the above-described method is applicable to any situation where rare event detection is necessary. In particular, the invention can be applied in any situation where a signal from a rare cell is to be detected where the rare cell is present at a concentration no greater than one rare cell for every 10,000 other cells. The invention is particularly applicable to those circumstances where the rare cell can be distinguished phenotypically from the other cells whereby the rare cell first is identified using a first signal, and then the genetic characteristics of the cell identified are determined using a second signal.

Any chromosomal abnormality or Mendelian trait could be diagnosed using the present rare cell technology. The only prerequisite is knowledge of the underlying molecular defect. Use of single fluorophores for the tagging of an individual allele creates an upper limit as to the number of mutations that can be tested simultaneously, however use of combinatorial chemistry increases enormously the number of allele specific mutations that can be tagged and detected simultaneously. Chromosomal abnormalities that fall within the scope of the invention include but are not limited to Trisomy 21, 18, 13 and sex chromosome aberrations such as XXX, XXY, XYY. With the use of combinatorial chemistry, the methods of the invention can be used to diagnose a multitude of translocations observed in genetic disorders and cancer. Mendelian disorders that fall within the scope of the invention include but are not limited to cystic fibrosis, hemochromatosis, hyperlipidemias, Marfan syndrome and other heritable disorders of connective tissue, hemoglobinopathies, Tay-Sachs syndrome or any other genetic disorder for which the mutation is known. The use of combinatorial chemistry dyes allows for the simultaneous tagging and detection of multiple alleles thus making it possible to establish the inheritance of predisposition of common disorders, e.g. asthma and/or the presence of several molecular markers specific for cancers, e.g., prostate, breast, colon, lung, leukemias, lymphomas, etc.

The invention is described in connection with observing “monolayers” of cells. Monolayer has a specific meaning as used herein. It does not require confluence and can involve single cell suspensions. It means simply that the cells are arranged whereby they are not stacked on top of one another, although all of the cells can be separated from one another. Thus, monolayers can be smears of single cell suspensions or can be a thin layer of tissue. Any solid or exfoliative cytology technique can be employed.

The invention also has been described in connection with identifying a pair of signals, one which identifies a target rare cell such as a fetal cell and another which is useful in evaluating the state of the cell such as a fetal cell having a genetic defect. It should be understood that according to certain embodiments, only a single signal need be detected. For example, where a fetal cell carries a Y chromosome and the diagnosis is for an abnormality on the Y chromosome, then the signal which identifies the genetic abnormality can be the same as that which identifies the fetal cell. As another example, a single signal can be employed in circumstances where the observed trait is a recessive trait. A pair of signals also can be used to detect the presence of two alleles or the existence of a condition which is diagnosed by the presence of two or more mutations in different genes. In these circumstances the pair of signals (or even several signals) can identify both the phenotype and the cell having that phenotype. Such embodiments will be apparent to those of ordinary skill in the art.

6. EXEMPLARY EMBODIMENTS

6.1. Smear Preparation

Smears were prepared from 10 .mu.l aliquots of whole blood on glass microscope slides. Smears were prepared from both cord blood and maternal circulating blood and allowed to air dry.

6.1.1. Cell Fixation

Fixation of smears prior to cell permeabilization for in situ PCR or PCR in situ hybridization was under one of three conditions. (i) Smears were fixed in ice-cold methanol for 10 minutes-16 hours. (ii) Smears were fixed in ice-cold 10% buffered formalin for 10 minutes-16 hours. (iii) Smears were fixed in 2% paraformaldehyde for 10 minutes-16 hours. Following fixation, smears were washed three times in phosphate buffered saline, at room temperature, for 10 minutes. Smears were then air-dried.

6.1.2. Cell Staining

Polychrome Staining:

The smears were covered with Wright's stain and incubated for one to two minutes at room temperature. Distilled water (2.5 ml) was then added to dilute the stain and incubation at room temperature continued for 3-6 minutes. The stain was then washed off rapidly with running water and a 1:10 dilution of Giemsa stain added to the slide. Incubation was at room temperature for 5 minutes and the stain was then washed off rapidly with running water. The smears were then air-dried.

Antibody Staining:

The smears were covered with anti-embryonal hemoglobin (hemoglobin .epsilon.-chain) monoclonal antibody and incubated at room temperature for one to three hours. The slides were then washed twice in phosphate buffered saline, at room temperature, for 5 minutes. Secondary antibody (anti-mouse antibody conjugated to phycoerythrin) was then added and the slide incubated at 37.degree. C. for 30 minutes. The slides were then washed twice in phosphate buffered saline, at room temperature, for 5 minutes and air-dried.

Fetal Hemoglobin Staining:

Smears were fixed in 80% ethanol for 5 to 10 minutes, then rinsed with tap water and air dried. Acid citrate-phosphate buffer (37.7 ml 0.1M citric acid, 12.3 ml 0.2M Na.sub.2HPO.sub.4, pH 3.3) was pre-warmed in a coplin jar in a 37.degree. C. water bath. The fixed smears were then added to the coplin jar and incubated at 37.degree. C. for 5 minutes. The smears were then rinsed with tap water and stained with 0.1% hematoxylin for one minute. The smears were then rinsed with tap water and stained with 0.1% eosin for one minute. The smears then underwent a final rinse in tap water and were air-dried.

Cell Permeabilization:

Cell permeabilization was attained by incubation in either proteinase K (1-5 mg/ml in phosphate buffered saline) or pepsin (2-5 mg/ml in 0.01M hydrochloric acid). Incubation was at room temperature for 1-30 minutes. Following permeabilization, smears were washed in phosphate buffered saline, at room temperature, for 5 minutes, then in 100% ethanol, at room temperature, for one minute. Smears were then air-dried.

PCR In Situ Hybridization:

For PCR in situ hybridization, smears were overlaid with 50 .mu.l amplification solution. Amplification solution comprised 10 mM Tris-HCl, pH 8.3, 90 mM potassium chloride, 1-5 mM magnesium chloride, 200 .mu.M dATP, 200 .mu.M dCTP, 200 .mu.M dGTP, 200 .mu.M dTTP, 1 .mu.M forward primer, 1 .mu.M reverse primer and 5-10 units thermostable DNA polymerase in aqueous sealing reagent. A glass coverslip was then lowered onto the amplification solution and the slide transferred to a thermal cyler. Following an initial denaturation step at 94.degree. C. for 4 minutes, the slide was then subjected to 25-35 cycles of amplification, where each cycle consisted of denaturation at 94.degree. C. for one minute, annealing at 55.degree. C. for one minute and extension at 72.degree. C. for one minute. The coverslip was then removed by incubation of the slide in phosphate buffered saline for 10 minutes at room temperature, and the slide air-dried. Fluorescein labeled oligonucleotide probe in hybridization buffer (600 mM sodium chloride, 60 mM sodium citrate, 5% dextran sulfate, 50% formamide) was then added and the slide covered with a glass cover slip, and incubated at 94.degree. C. for 10 minutes then at 37.degree. C. for one hour. The coverslip was then removed by incubation of the slide in phosphate buffered saline for 10 minutes at room temperature and the slide then washed twice for 5 minutes in phosphate buffered saline at room temperature. The smear was then covered with protein block solution (1% bovine serum, 2.5% goat serum, 0.2% Tween-20) and incubated at room temperature for 10 minutes. The solution was then removed and the slide washed three times in phosphate buffered saline for 5 minutes at room temperature. The smear was then covered with mouse anti-fluorescein monoclonal antibody and incubated at room temperature for 20 minutes. The solution was then removed and the slide washed three times in phosphate buffered saline for 5 minutes at room temperature. The smear was then covered with biotinylated goat anti-mouse F(ab).sub.2 and incubated at room temperature for 20 minutes. The solution was hen removed and the slide washed three times in phosphate buffered saline for 5 minutes at room temperature. The smear was then covered with alkaline phosphatase conjugated streptavidin and incubated at room temperature for 20 minutes. The solution was then removed and the slide washed twice in phosphate buffered saline for 5 minutes at room temperature. Alkaline phosphatase substrate solution (50 mg/ml BCIP, 75 mg ml NBT) was then added to the smear and the slide incubated at 37.degree. C. for 10 minutes-two hours. The slide was then washed twice in distilled water at room temperature for 5 minutes and air-dried.

In Situ PCR:

For in situ PCR, smears were overlaid with 50 l amplification solution. Amplification solution comprised 10 mM Tris-HCl, pH 8.3. 90 mM potassium chloride, 15 mM magnesium chloride, 200 .mu.M dATP, 200 .mu.M dCTP, 200 .mu.M dGTP, 0.5 .mu.M [R110]dUTP, 1 .mu.M forward primer, 1 .mu.M reverse primer and 5-10 units thermostable DNA polymerase in aqueous sealing reagent. A glass coverslip was then lowered onto the amplification solution and the slide transferred to a thermal cycler. Following an initial denaturation step at 94.degree. C. for 4 minutes, the slide was then subjected to 25-35 cycles of amplification, where each cycle consisted of denaturation at 94.degree. C. for one minute, annealing at 55.degree. C. for one minute and extension at 72.degree. C. for one minute. The coverslip was then removed by incubation of the slide in phosphate buffered saline for 10 minutes at room temperature and the slide air-dried.

6.2. Automated Smear Analysis

Automated smear analysis has been briefly summarized, above. The apparatus and method used in the exemplary embodiment is now described.

6.2.1. Apparatus

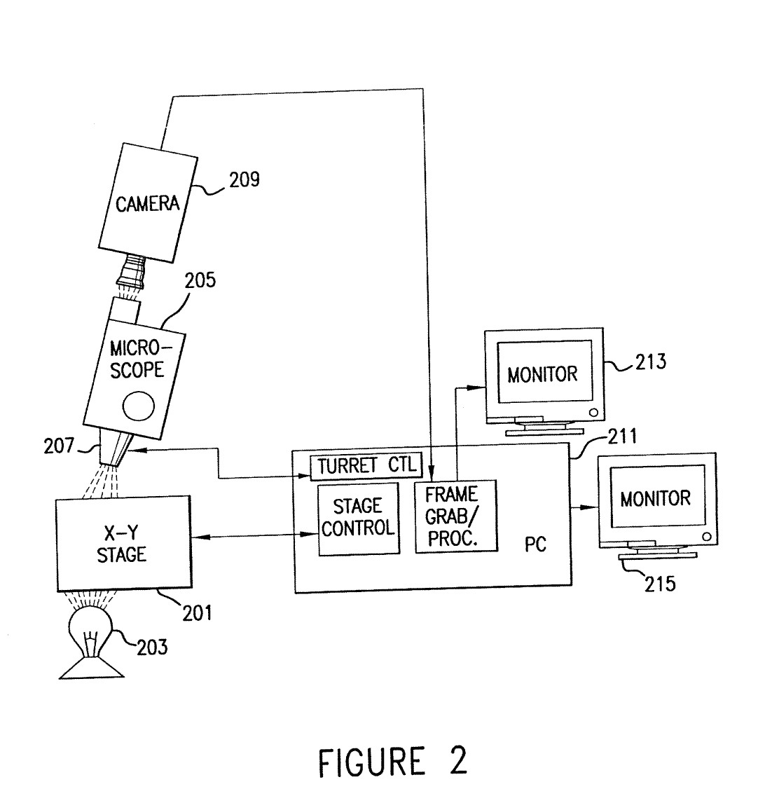

The block diagram of FIG. 2 shows the basic elements of a system suitable for embodying this aspect of the invention. The basic elements of the system include an X-Y stage 201, a mercury light source 203, a fluorescence microscope 205 equipped with a motorized objective lens turret (nosepiece) 207, a color CCD camera 209, a personal computer (PC) system 211, and one or two monitors 213, 215.

The individual elements of the system can be custom built or purchased off-the-shelf as standard components. Each element will now be described in somewhat greater detail.

The X-Y stage 201 can be any motorized positional stage suitable for use with the selected microscope 205. Preferably, the X-Y stage 201 can be a motorized stage that can be connected to a personal computer and electronically controlled using specifically compiled software commands. When using such an electronically controlled X-Y stage 201, a stage controller circuit card plugged into an expansion bus of the PC 211 connects the stage 201 to the PC 211. The stage 201 should also be capable of being driven manually. Electronically controlled stages such as described here are produced by microscope manufacturers, for example including Olympus (Tokyo, Japan), as well as other manufacturers, such as LUDL (NY, USA).

The microscope 205 can be any fluorescence microscope equipped with a reflected light fluorescence illuminator 203 and a motorized objective lens turret 207 with a 20.times. and an oil immersion 60.times. or 63.times. objective lens, providing a maximum magnification of 600.times. The motorized nosepiece 207 is preferably connected to the PC 211 and electronically switched between successive magnifications using specifically compiled software commands. When using such an electronically controlled motorized nosepiece 207, a nosepiece controller circuit card plugged into an expansion bus of the PC 211 connects the stage 201 to the PC 211. The microscope 205 and stage 201 are set up to include a mercury light source 203, capable of providing consistent and substantially even illumination of the complete optical field.

The microscope 205 produces an image viewed by the camera 209. The camera 209 can be any color 3-chip CCD camera or other camera connected to provide an electronic output and providing high sensitivity and resolution. The output of the camera 209 is fed to a frame grabber and image processor circuit board installed in the PC 211. A camera found to be suitable is the SONY 930 (SONY, Japan).

Various frame grabber systems can be used in connection with the present invention. The frame grabber can be, for example a combination of the MATROX IM-CLD (color image capture module) and the MATROX IM-640 (image processing module) set of boards, available from MATROX (Montreal, CANADA). The MATROX IM-640 module features on-board hardware supported image processing capabilities. These capabilities compliment the capabilities of the MATROX IMAGING LIBRARY (MIL) software package. Thus, it provides extremely fast execution of the MIL based software algorithms. The MATROX boards support display to a dedicated SVGA monitor. The dedicated monitor is provided in addition to the monitor usually used with the PC system 211. Any monitor SVGA monitor suitable for use with the MATROX image processing boards can be used. One dedicated monitor usable in connection with the invention is a ViewSonic 4E (Walnut Creek, Calif.) SVGA monitor.

In order to have sufficient processing and storage capabilities available, the PC 211 can be any INTEL PENTIUM-based PC having at least 32 MB RAM and at least 2 GB of hard disk drive storage space. The PC 211 preferably further includes a monitor. Other than the specific features described herein, the PC 211 is conventional, and can include keyboard, printer or other desired peripheral devices not shown.

6.2.2. Method

The PC 211 executes a smear analysis software program compiled in MICROSOFT C++ using the MATROX IMAGING LIBRARY (MIL). MIL is a software library of functions, including those which control the operation of the frame grabber 211 and which process images captured by the frame grabber 211 for subsequent storage in PC 211 as disk files. MIL comprises a number of specialized image processing routines particularly suitable for performing such image processing tasks as filtering, object selection and various measurement functions. The smear analysis software program runs as a WINDOWS 95 application. The program prompts and measurement results are shown on the computer monitor 213, while the images acquired through the imaging hardware 211 are displayed on the dedicated imaging monitor 215.

In order to process microscopic images using the smear analysis program, the system is first calibrated. Calibration compensates for day to day variation in performance as well as variations from one microscope, camera, etc., to another. During this phase a calibration image is viewed and the following calibration parameters are set:

the color response of the system;

the dimensions or bounds of the area on a on a slide containing a smear to be scanned for fetal cells;

the actual dimensions of the optical field when using magnifications 20.times. and 60.times. (or 63.times.); and

the minimum and maximum fetal nuclear area when using magnifications 20.times. and 60.times. (or 63.times.).

6.2.3. Detection of the First (Identification) Signal

The fetal cell detection algorithm operates in two stages. The first is a pre-scan stage I, illustrated in the flow chart of FIG. 3, where possible fetal cell positions are identified using a low magnification and high speed. The 20.times. objective is selected and the search of fetal cells can start:

The program moves the automated stage (FIG. 2, 201) to a preset starting point, for example one of the corners of a slide containing a smear (Step 301).

The x-y position of the stage at the preset starting point is recorded (Step 303) optical field.

The optical field is acquired (Step 305) using the CCD camera 209 and transferred to the PC 211 as an RGB (Red/Green/Blue) image.

The RGB image is transformed (Step 307) to the ILLS (Hue/Luminance/Saturation) representation.

The Hue component is binary quantized (Step 309) as a black and white image so that pixels with Hue values ranging between 190 and 255 are set to 0 (black) representing interesting areas (blobs), while every other pixel value is set to 255 (white, background). The blobs represent possible fetal cell nuclear areas.

The area of each blob in the binary quantized image is measured. If, at 20.times. magnification, it is outside a range of about 20 to 200 pixels in size, the blob's pixels are set to value 255 (background); they are excluded from further processing (Steps 311, 313, 315 and 317).

Then the coordinates of each blob's center of gravity (CG) are calculated (step 319), using a custom MATROX function. The center of gravity of a blob is that point at which a cut-out from a thin, uniform density sheet of material of the blob shape would balance. These coordinates are stored in a database along with the z-y position of the current optical field, so the blob can be located again at the next processing stage using higher magnification.

Additional optical fields are processed similarly recording the x-y position of each succeeding optical field, until the complete slide are is covered (Steps 321 and 323).

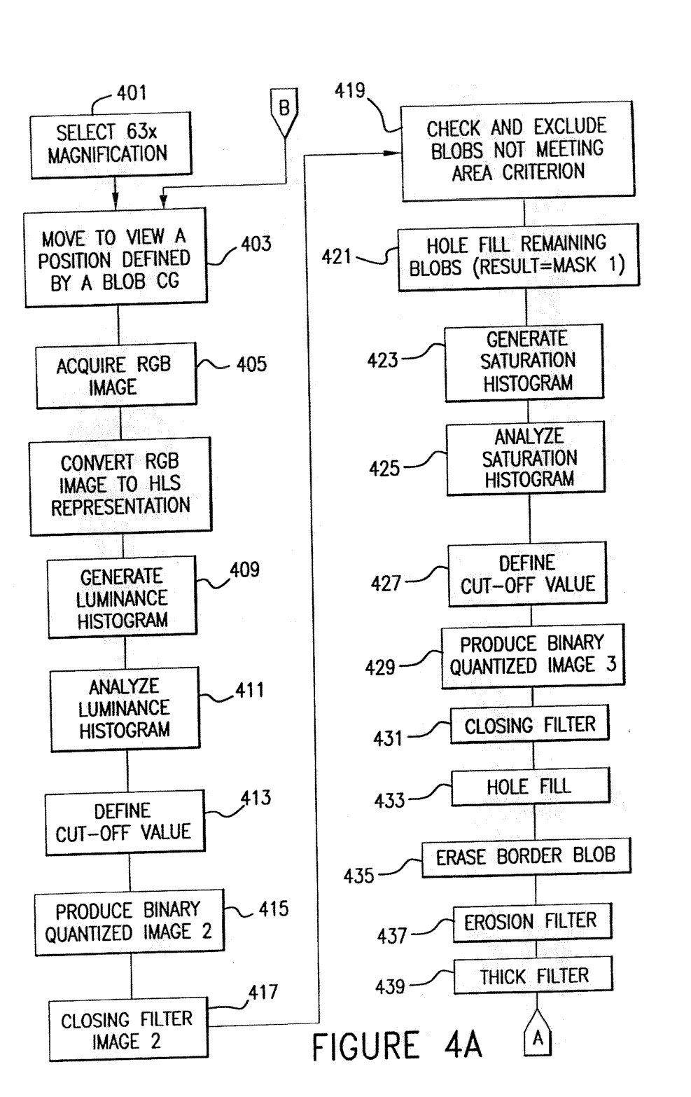

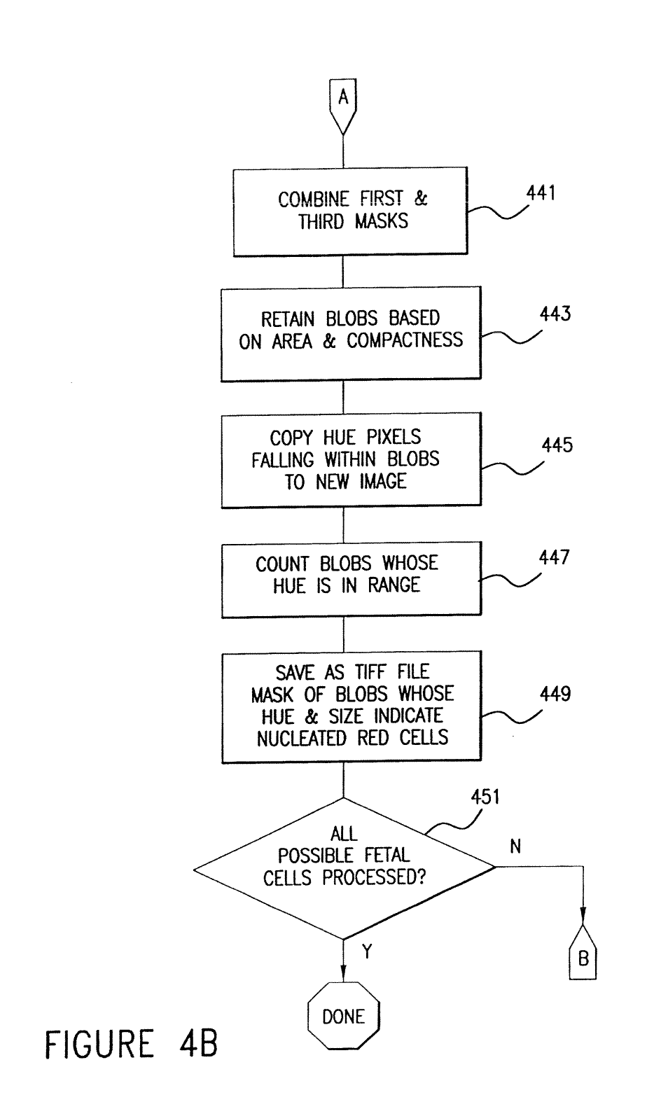

Stage II, illustrated in the flow chart of FIGS. 4A and 4B, includes the final fetal cell recognition process:

63.times. magnification is selected (Step 401).

The program moves the automated stage (FIG. 2, 201) so that the coordinates of the first position of a CG found earlier, which is possible fetal cell nuclear area, is at the center of the optical field (Step 403).

The optical field is acquired using the CCD camera (FIG. 2, 209) and transferred to the computer as an RGB image (Step 405).

The RGB image is transformed to the HLS model (Step 407).

The program then generates a Luminance histogram (Step 409) by counting the number of pixels whose Luminance value equals each possible value of Luminance. The counts are stored as an array of length 256 containing the count of pixels having a gray-level value corresponding to each index into the array.

The program next analyzes the Luminance distribution curve (Step 411), as represented by the values stored in the array, and locates the last peak. It has been found that this peak includes pixel values that represent plasma area in the image. The function that analyzes the Luminance distribution curve: calculates a 9-point moving average to smooth the curve; calculates the tangents of lines defined by points 10 grey-level values distant; calculates the slopes of these lines in degrees;

finds the successive points where the curve has zero slope and sets these points (grey-levels) as −1 if they represent a minimum (valley in the curve) or 1 if they represent a maximum (peak in the curve); then finds the locations of peaks or valleys in the curve by finding the position of a 1 or a −1 in the array of grey-level values.

The program then sets as a cut-off value the grey-level value of pixels lying in the valley of the Luminance distribution which occurs before the last peak of the distribution (Step 413).

Using this cut-off value, the program then produces (Step 415) a second binary quantized image. This is a black-and-white image in which pixels corresponding to pixels in the Luminance image having grey-level values lower than the cut off point are set to 255 (white) and pixels corresponding to pixels in the Luminance image having grey-level values higher than the cut off point are set to 0 (black). The white blobs of this image are treated as cells while the black areas are treated as non-cellular area.

A closing filter is applied (Step 417) to the second binary quantized image; in this way holes, i.e., black dots within white regions, are closed.

The program now measures the area of the cells. If the area of any of the cells is less than 200 pixels then these cells are excluded, i.e. the pixels consisting these cells are set to pixel value 255 (black) (Step 419).

A hole fill function, found in the MIL, is applied to the remaining blobs (Step 412). The resulting binary quantized image, after processing, is a mask whose white regions denote only cells.

Red blood cells are now distinguished from white blood cells based on the Saturation component of the HLS image. The mask is used to limit processing to only the cell areas.

The program now counts the number of pixels whose Saturation value is each possible value of Saturation. The counts are stored as an array of length 256 containing the count of pixels having a grey-level value corresponding to each index into the array (Step 423).

The program now analyzes (Step 425) the Saturation distribution curve, as represented by the values stored in the array, and locates the first peak. This peak includes pixel values that represent areas contained in white blood cells.

The grey-level value that coincides with the first minimum (valley) after the peak is set as a cut-off point (Step 427).

Using this cut-off value the program produces (Step 429) a third binary quantized image. Pixels corresponding to pixels in the Saturation image having grey-level values higher than the cut-off point are set to 255 (white). They constitute red blood cell areas. Pixels corresponding to pixels in the Saturation image having grey-level values lower than the cutoff point are set to 0 (black). The white blobs of this third binary quantized image are seeds for areas that belong to red blood cells.

A closing filter is applied (Step 431) to the third binary quantized image; in this way holes, i.e., black dots within white regions, are closed.

A hole fill function, found in the MIL, is applied (Step 433) to the remaining blobs. The resulting binary quantized image, after processing, is a new mask that contains only white blood cells.

An erase border blob function of MIL is now applied (Step 435) to the remaining blobs, removing those which include pixels coincident with a border of the image area. Such blobs cannot be included in further processing as it is not known how much of the cell is missing when it is coincident with a border to the image area.

An erosion filter is applied 6 times to this mask; thus any connected blobs (white blood cell seeds) are disconnected (Step 437).

A “thick” filter is applied 14 times (Step 439). The “thick” filter is equivalent to a dilation filter. That is, it increases the size of a blob by successively adding a row of pixels at the periphery of the blob. If a growing blob meets an adjacent blob growing next to it, the thick filter does not connect the two growing blobs. Thus adjacent blobs can be separated.

The first binary quantized mask (containing all the cells) and the third binary quantized mask (containing the separated seeds of white blood cells) are combined with a RECONSTRUCT_FROM_SEED MIL operator. A fourth mask thus constructed contains blobs (cells) copied from the first mask that are allowed by the third mask and therefore represent white blood cells (Step 441).

The blobs in the fourth mask are measured for their area and compactness: Area (A) is the number of pixels in a blob; Compactness is derived from the perimeter (p) and area (A) of a blob, it is equal to: p2/4(A). The more convoluted the shape, the bigger the value. A circle has the minimum compactness value (1.0). Perimeter is the total length of edges in a blob, with an allowance made for the staircase effect which is produced when diagonal edges are digitized (inside corners are counted as 1.414, rather than 2.0). Blobs are retained in the fourth mask only if their area is between 1000 and 8000 pixels and they have a compactness less than 3, thus allowing for cells with relatively rough outline. Blobs that touch the border of the image are excluded from further processing (Step 443).

The fourth mask is applied to the Hue component in the following manner (Steps 445, 447, 449 and 451):

Pixels from the Hue component are copied to a new image retaining their Hue value, provided that their coordinates coincide with white (255) pixels in the “mask”; all other pixels in the new image are set to 0 (black) (Step 445).

The pixel values in each of the contiguous non-0 pixel areas, i.e., those blobs corresponding to images of red cells, are checked for values between 190 and 255. The number of such pixels in each blob is counted (Step 447).

If there are more than 200 such pixels, the blob represents a nucleated red blood cell. The coordinates of the center of gravity of each such cell are stored. The mask is binary quantized so that all pixels having non-0 values are set to 255 (white); and the mask is stored as a separate Tagged Image File Format (TIFF) file (Step 449).

The program moves to the next stored coordinates for a possible fetal cell which do not coincide with any of the coordinates stored during the previous step. The entire process is repeated until a preset number of nucleated red blood cells have been identified. The results, including the nucleated red blood cell coordinates and the names of the respective mask files, along with various characteristic codes for the blood slide are stored in a result text file. The nucleated red blood cells whose coordinates are stored are the fetal cells sought (Step 451).

After fetal cells are identified, the second signal is generated, for example by in situ CR or PCR in situ hybridization or FISH, as described above.

6.2.4. Detection of the Second Signal

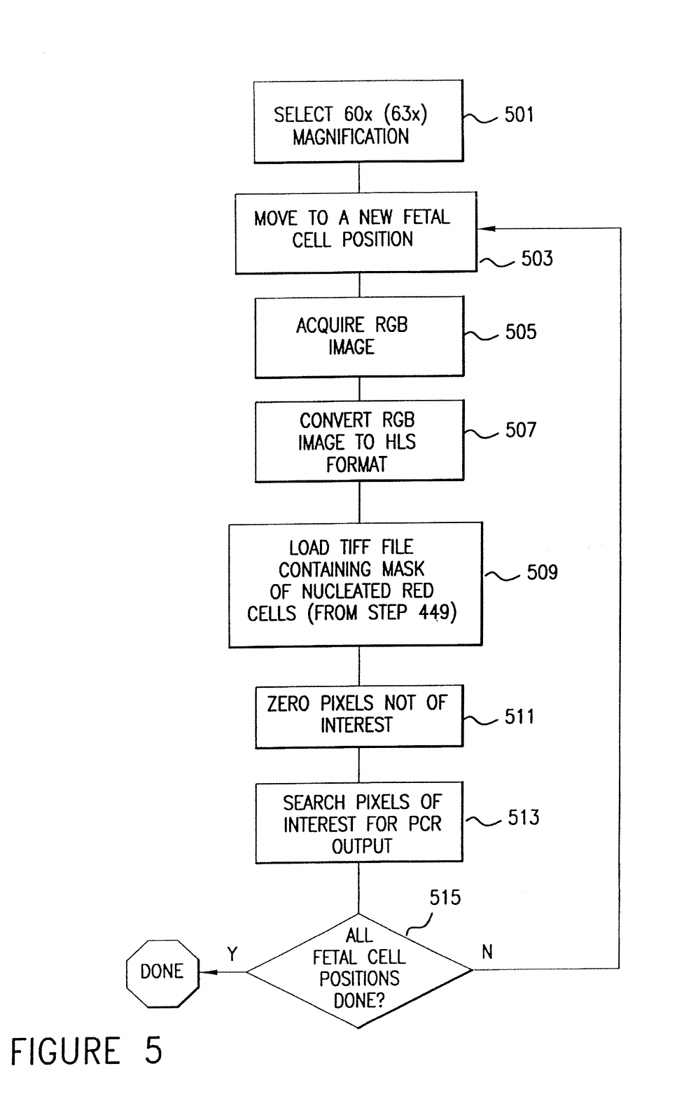

A smear including in situ PCR or PCR in situ hybridization treated cells is positioned on the stage (FIG. 2, 201). If necessary calibration steps are taken, as before. Calibration permits the software to compensate for day to day variation in performance as well as variations from one microscope, camera, etc. to another. Detection of the second signal then proceeds, as shown in the flow chart of FIG. 5, as follows:

Magnification objective 60.times. (63.times.) is chosen (Step 501).

The x-y stage is moved to the first fetal cell position according to data from the result file compiled from detection of the first signal, as described above (Step 503).

The optical field is acquired using the CCD camera (FIG. 2, 209) and transferred to the computer (FIG. 2, 211) as an RGB image (Step 505).

The RGB image is transformed to the HLS model (Step 507).

The TIFF file containing the black and white mask is loaded as a separate image (Step 509).

The pixels of the Hue component not corresponding to white areas in the mask arc set to 0 (black) (Step 511).

The remaining areas, which represent fetal cells, are searched for pixel values corresponding to a signal produced following PCR. For example, the signal may be a color which arises due to the presence of alkaline phosphatase, i.e., red. The non black areas of the Hue component are searched for pixel values ranging from 0 to 30 (Step 513).

The stage is moved to the next non-processed fetal cell and the above process is repeated (Step 515).

6.3. Variations

A number of variations on the above-described system and method are also contemplated and are encompassed by the present invention. Some of these are now described. This description will also suggest others to those skilled in the art.

Each unenriched blood sample may be used to prepare smears on each of a plurality of individual microscope slides. When prepared in this way, each slide can undergo detection of the first signal. However, only those slides which the first signal is detected need be further processed to generate the second signal, and subsequently are analyzed to detect the second signal. Processing in this way permits the use of conventional sample and slide-handling equipment.

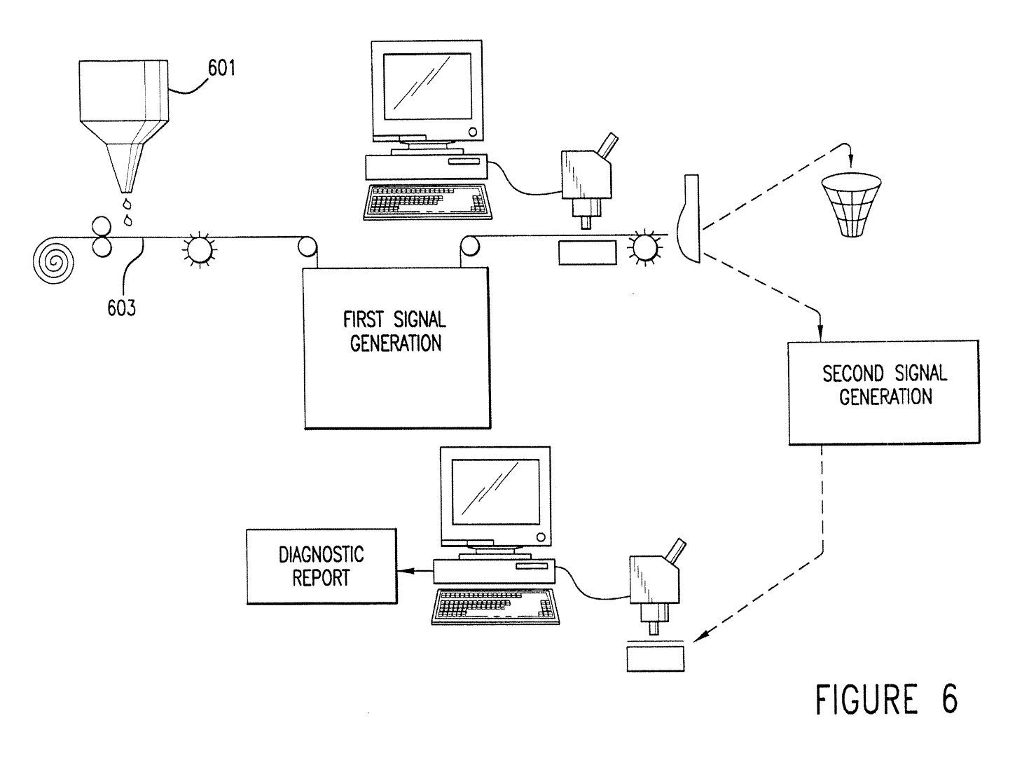

In a variation illustrated schematically in FIG. 6, the unenriched blood sample 601 is used to prepare a single, long smear on a flexible substrate 603. The substrate 603 can have a length 10 or more times its width. For example, a strip of cellulose acetate film base with sprocket holes on either side could be used as the substrate. The strip carrying the smear undergoes the processing steps described above in a continuous processing system, as shown in FIG. 6. After locations of fetal cells are determined by detection of the first signal, segments of the smear including those locations are cut out of the continuous strip for generation and detection of the second signal.

In an alternative processing method using a single, long smear on a flexible substrate, the strip is divided into a plurality of individual segments similar to microscope slides, before generating and detecting the first signal. Processing proceeds as for individual microscope slides.

The above variations, and similar variations, are advantageous in that the entire smear need not be processed for generation and detection of the second signal. Only those slides or segments in which the first signal is detected need undergo the further processing to generate and detect the second signal.

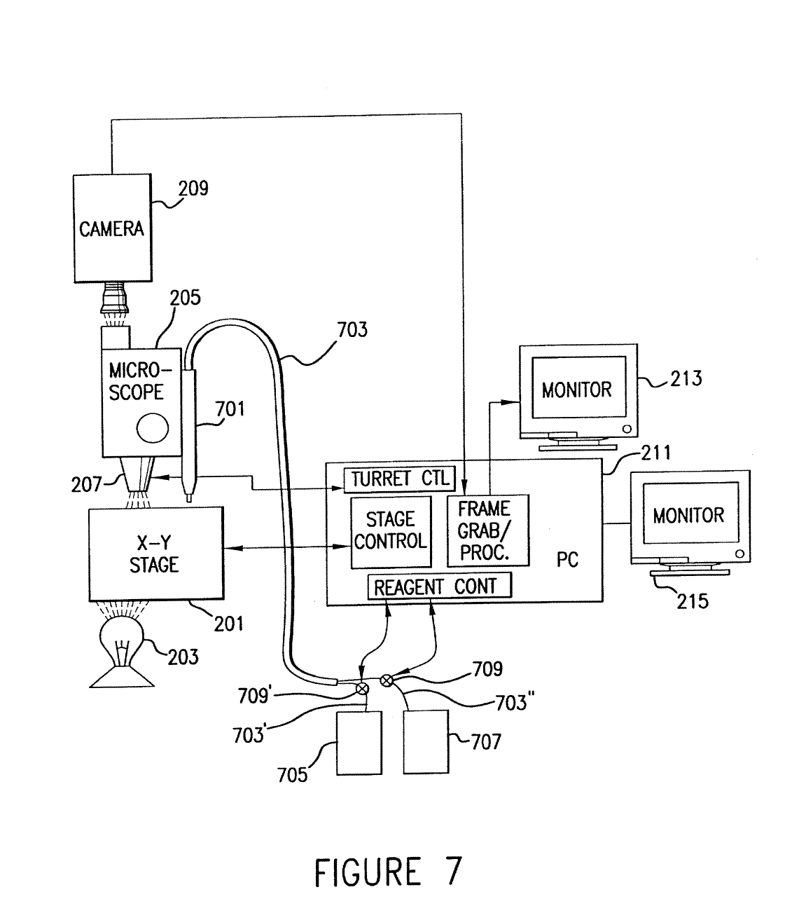

In one aspect of the invention, a device is provided for dispensing reagents only to those portions of the smear where a rare cell is detected. Referring to FIG. 7, an apparatus of the invention is shown including a reagent dispenser system. The reagent dispenser system can be located for dispensing reagents to precise locations on the stage. This is particularly suited for dispensing reagents only of the coordinates identified by a first signal, such as the coordinates of a rare cell (e.g., a fetal cell and a maternal blood smear). The system includes a reagent dispenser 701 which is a housing for one or more micropipettes located within the housing. The reagent dispenser is attached in this embodiment to the microscope and is positioned relative to the stage in fixed relation to the microscope. The narrow tip of the reagent dispenser 701 is adjacent the stage 201. The opposite end of the reagent dispenser 701 has communicating therewith feedline 703 which is a tube or a housing carrying a plurality of tubes for delivering reagents to the reagent dispenser 701. The feedline 703 is attached remote from the reagent dispenser 701 to a first reagent container 705 and a second reagent container 707. In the embodiment shown, the feedline 703 is a housing through which passes feedline 703′ communicating with reagent container 705 and feedline 703′ communicating with reagent container 707. A pump 709 is attached to feedline 703″ for pumping reagent from the reagent container 707 to the reagent dispenser 701, and out the narrow tip of the reagent dispenser 701 onto the stage at a desired location. Another pump 709′ is attached to feedline 703′ for delivering reagents from reagent container 705 to the reagent dispenser 701. The pumps are electronically controlled by PC 211 using specifically compiled software commands indicated by “reagent control”. The reagents can be any one of the reagents described above in connection with generating a signal.

In the embodiment shown, the reagent dispenser is attached to the microscope. The reagent dispenser need not be attached to the microscope and, instead, can be otherwise attached to any frame relative to the X-Y stage. The stage is shown as moving with respect to the reagent dispenser for locating the narrow tip of the reagent dispenser at a precise location with respect to a slide on the stage. The slide on the stage can be moved to a different location, and the reagent dispenser can be itself moveably controlled to locate it relative to a set of coordinates in the slide. What is important is that, in an automated fashion, the coordinates of a detected rare cell can be positioned with respect to the dispensing end of the reagent dispenser, whereby materials may be delivered to a discrete location at the coordinates of the rare cell. If the reagent dispenser is controlled by a motor and moveable with respect to a stage or a slide upon a stage, then the reagent dispenser can be provided with a sensor for locating its position with respect to the slide or stage. Thus, the slide on a stage can be processed in series, with the microscope first locating the coordinates on the slide of the rare cell. The slide then is next moved to a second processing area where the reagent dispenser is positioned at the previously-identified coordinates in the slide and reagents are delivered to generate the second signal. Optionally the slide could be moved to a third station, such as a thermocycling station and then back to the microscope field for viewing.

It should be evident that different treatments of the smear are possible when it is desired to identify a different cell type or to diagnose a different cellular characteristic. The biochemistry, morphological parameters and colors described above may each be varied in known ways to meet other diagnostic needs.

The methods of the invention provide convenient, sensitive, automatable procedures for assessing the state of ploidy in fetal cells derived from maternal blood. Any finding of aneuploidy in a nucleus carries a presumptive conclusion that the cell from which the nucleus originated was fetal in origin, since it is presumed or has been verified that the mother's genetic background is euploid with respect to the chromosome in question. The ability to automate the analysis means that, if necessary or desired, more than one slide from a given specimen may be prepared, thus increasing the dynamic range for finding a small number of potential aneuploid-positive cells in a sample of maternal blood that contains preponderantly maternal cells. The inventors believe this represents an unanticipated advance over the state of the art.

Automated apparatuses and methods for carrying out the microscopic analysis of biological samples enhance diagnostic procedures and optimize the throughput of samples in a microscope-based diagnostic facility. A robotic microscope system is described in co-owned U.S. patent application Ser. No. 11,833,203 filed Aug. 2, 2007. Among its disclosures, an integrated microscope system displaceable along a second surface is provided. The integrated microscope system includes an automated robotic microscope system housed in a light-tight enclosure. In this system, the automated robotic microscope system includes (i) a microscope having a stage; (ii) at least one specimen slide positionable on the stage; (iii) a light source that illuminates the slide; (iv) an image capture device that captures an image of the specimen; and (v) electrical, electronic and/or computer-driven means communicating with and controlling positioning of said specimen slide, said light source, and said image capture device. Furthermore, in this system the light-tight enclosure includes at least one shelf interior to said enclosure, wherein said automated robotic microscope system is positioned on a shelf; and a viewing monitor disposed in a surface of said enclosure viewable from a location exterior to the enclosure.

A dynamic automated microscope operation and slide scanning system is described in co-owned U.S. patent application Ser. No. 11,833,594 filed Aug. 3, 2007. Embodiments disclosed include an automated microscope and method for dynamically scanning a specimen mounted on a microscope slide using a dynamic scanning microscope incorporating a microscope slide stage, at least one source of illumination energy, at least one electronic imaging device, at least one interchangeable component carousel and a synchronization controller. An exemplary automated microscope has the ability to significantly reduce the time required to perform an examination, reduce vibration reaching the system, and to provide diagnostic results. During the imaging process, the stage and color filter wheel are in constant motion rather than stationary as in previous approaches. Real time position sensors on each of the moving sub-systems accurately telemeter the instant position of the stage mounted slide and the color filter wheel. The color filter wheel rotates at a sufficient speed to allow the capture of images, at each of the filter wavelengths, at each imaging location and focal plane.

Interchangeable objective lenses, filters, and similar elements for use in an automated microscope system are described in co-owned U.S. patent application Ser. No. 11,833,154 filed Aug. 2, 2007. This application generally relates to remotely operated or robotically controlled microscopes, and specifically to the mechanization of a means for automatically interchanging objective lens assemblies, filters and/or other optical components. An apparatus for interchanging optical components in an optical path is disclosed, which includes a control motor having a rotatable motor shaft; a support structure supporting the control motor; a planar base defined by a periphery that is generally symmetric about a central point on the planar base, the planar base including a plurality of mounting fixtures housing a plurality of optical components equi-angularly placed at a same distance from the base center, and a mechanism that causes generally symmetric rotation of the planar base about its center, so that a particular optical component of choice is positioned in the optical beam

An automated microscope stage for use in an automated microscope system is described in co-owned U.S. patent application Ser. No. 11,833,183 filed Aug. 2, 2007. This application generally relates to a microscope stage that is adjustably moveable along the optic axis of the microscope. For example, a microscope slide mount is disclosed that is adjustable along a direction of the optic axis of the microscope, including a base plate; a microscope stage assembly movably mounted on said base plate operably configured to permit displacement of the assembly along the direction of the optic axis; and a microscope slide holding means fixed to said microscope stage assembly.

An automated microscope slide cassette and slide handling system for use in an automated microscope system is disclosed in co-owned U.S. patent application Ser. No. 11,833,517 filed Aug. 3, 2007. This application discloses a mechanism for removing and replacing a slide housed in a cassette defining a plurality of slots configured for holding slides in spaced parallel configuration.

An automated microscope slide loading and unloading mechanism for use in an automated microscope system is described in co-owned U.S. patent application Ser. No. 11,833,428 filed Aug. 3, 2007. An exemplary embodiment discloses a microscope slide manipulation device which includes: a base structure; a sleeve defining a through-void, the sleeve having a first end and a second end, the second end fastened to the base, and the sleeve being oriented perpendicular to the base; a longitudinal shaft symmetric about an imaginary longitudinal axis in part positioned in the sleeve through-void in a manner to permit axial and longitudinal movement of the longitudinal shaft in the sleeve through-void, the longitudinal shaft having a shaft first end and a shaft second end, the shaft second end positioned within the sleeve through-void and the shaft first end projecting beyond the sleeve first end and including a parallel track structure in a plane to the sleeve imaginary longitudinal axis; a plate slideably positioned between the parallel track structures on the sleeve first end, the plate having a first plate end and a second plate end, one of the first plate end or second plate end having a two-pronged forked configuration defining a void area between each prong that corresponds to the width of a microscope slide, and wherein the fork has a gripping structure operatively configured to permit gripping of a microscope slide along its edges.

Automated methods that employ computer-resident programs to drive the microscopic detection of fluorescent signals from a biological sample, useable to drive an automated microscope system, are disclosed in co-owned U.S. patent application Ser. No. 11,833,849 filed Aug. 3, 2007. An exemplary method of microscopic analysis, adaptable for high throughput analysis of multiple samples, disclosed therein includes steps of providing an automated microscope comprising a slide stage, at least one objective lens, image capturing means, programmable means for operating the microscope according to a protocol, and programmable means for providing an analytical outcome; providing a microscope slide containing a sample and interrogatable data thereon, wherein the interrogatable data provide information related to a protocol for analysis of said sample; interrogating the data; positioning the slide on the slide stage; causing the microscope to analyze the sample in accordance with the analytical protocol encoded in the interrogatable data; and causing the microscope to provide an analytical outcome representing the sample. Automatic operation of a microscope using computer-resident programs to drive the microscope in conducting a FISH assay for image processing is described in co-owned U.S. patent application Ser. No. 11,833,204 filed Aug. 2, 2007. Embodiments are disclosed which perform various image processing functions that may be employed to implement an automated fluorescence in situ hybridization method. The embodiments include an auto-exposure method for acceptably imaging all regions of the sample over an intensity range exceeding the dynamic range of the digital electronics; a method for enumeration of fluorescence in situ hybridization objects-of-interest which locates targets within the sample; nuclei identification which is a method for classifying and characterizing the objects-of-interest enumerated; segmenting nuclei which, is a method for defining the shape of an identified object of interest. Embodiments of the method are useful to characterize cell nuclei, or to enumerate a chromosome.

Methods disclosed herein are directed toward automating the detection and analysis of tissue specimens whose cells are suspected of harboring genes that have undergone somatic gene duplication or gene amplification during carcinogenesis. The methods afford computer driven image accumulation, and computer driven analysis of images obtained, as well as reporting results of such analyses in a variety of formats in an automated procedure that frees the methods from human intervention to a significant extent. Reports may be presented, by way of nonlimiting example, in the form of charts, tables, images of representations of a field on a slide, and the like. Reports are in digital formats as files or records, and as such are conveniently disseminated to local or remote locations for review. Because of the use of automated fluorescence microscopy, such as a system including components and software that is referenced herein, rapid, convenient, and accurate screening of tissue samples is afforded. These methods, and the automated microscope system employed in implementing them, are particularly well suited for use in high throughput analysis of a plurality of tissue samples.

Biological samples used in the instant methods may be derived from maternal blood samples. Other sources of fetal cells are also contemplated in the present methods. Such sources are manipulated, in various embodiments, to provide cell-free preparations of nuclei for deposition on microscope slides.

In various embodiments a slide-mounted sample is then treated with a generic fluorescent dye that stains chromosomes or nucleic acids with a fluorescent probe having a particular emission color isolatable by a suitable optical filter. A nonlimiting example of a generic dye is 4′,6-diamidino-2-phenylindole (DAPI). Staining with DAPI affords a means of identifying the location of nuclei, or of chromosomes, for the computer driven process of image capture for further capture of images from FISH probes.

The tissue specimen is hybridized to a fluorescently labeled FISH probe whose nucleotide sequence is constructed specifically to target a gene sequence, or a segment or portion of a gene sequence, that is specific for a potential aneuploid chromosome sought to be targeted. The various fluorescent labels used in the probes are optically isolatable by the use of suitable filters and related optical components. The specificity of the nucleotide sequence ensures that all, or most, chromosomes in a specimen having the target sequence are in fact hybridized to the probe, while non-target sequences remain unhybridized. Hybridization is caused to proceed by heating sufficiently to denature the target sequence, thereby exposing single stranded DNA complementary to the probe. The process then continues by annealing the probe to the exposed single strand, thus labeling the sequence with the fluorescent label. A worker of skill in the field of the invention knows specific conditions, such as solution ionic strength, buffer composition, temperature, and the like, to achieve the required hybridization. Following annealing the excess probe is rinsed away.

The slide bearing the hybridized specimen is inserted into a slide-loadings cassette that is a component of the automated microscope system. The system is set into operation, at which point the slide is caused to be transported from the cassette and placed on the stage of the microscope. In many embodiments each slide may bear a code interrogatable by the automated microscope that may include information such as a specimen identification, and the identities of any generic chromosome dye, and the various fluorescent labels on the FISH probes, used with the specimen in question. Such information guides the automated microscope in selection of appropriate optical filters and related optical elements for use throughout the image accumulation process.

Automated analysis may begin by directing the use of a low magnification of the microscope, using at least the generic dye, and possibly the probe labels, to identify regions within the specimen for imaging at a higher magnification. When the computer software identifies regions of interest at low magnification, it may direct the automated microscope to interchange objective lenses and/or filters, and any other optical components, for suitable image analysis of identified loci at higher magnification based on emitted light originating from one or another of a fluorescent label used in a probe. The computer software may then use features in an image, by way of nonlimiting example, the intensity and number of FISH-labeled spots, to enumerate such spots arising within single nuclei. Such an enumeration may provide a resulting indication of aneuploidy in fetal cells present in the specimen being analyzed.

Nucleic acid probes suitably labeled for use in FISH may be prepared by use of labeled mononucleoside triphosphates or their derivatives in enzyme catalyzed nucleic acid synthetic procedures, or by chemical synthesis. These procedures are widely known to workers of skill in the field of the invention. In particular, nucleic acid probes directed at detectable portions of various chromosomes, useful in establishing the state of ploidy of fetal cells are widely known in the field of the invention.

The FISH technique may be used for identifying chromosomal abnormalities and gene mapping. For example, a FISH probe to chromosome 21 permits one to label cells with trisomy 21, an extra chromosome 21, the cause of Down syndrome. FISH kits comprising multicolor DNA probes are commercially available. For example, AneuVysion Multicolor DNA Probe Kit sold by the Vysis division of Abbott Laboratories, is designed for in vitro diagnostic testing for abnormalities of chromosomes 13, 18, 21, X and Y in amniotic fluid samples via fluorescence in situ hybridization (FISH) in metaphase cells and interphase nuclei. The AneuVysion® Assay (CEP 18, X, Y-alpha satellite, LSI 13 and 21) Multi-color Probe Panel uses CEP 18/X/Y probe to detect alpha satellite sequences in the centromere regions of chromosomes 18, X and Y and LSI 13/21 probe to detect the 13q14 region and the 21q22.13 to 21q22.2 region. The combination of colors evidenced is used to determine whether there is normal chromosome numbers or trisomy.

Similarly, the UroVysion kit provided by the Vysis division of Abbott Laboratories is designed to detect chromosomal abnormalities associated with the development and progression of bladder cancer by detecting aneuploidy for chromosomes 3, 7, 17, and loss of the 9p21 locus via fluorescence in situ hybridization (FISH) in urine specimens from persons with hematuria suspected of having bladder cancer. The UroVysion Kit consists of a four-color, four-probe mixture of DNA probe sequences homologous to specific regions on chromosomes 3, 7, 9, and 17. The UroVysion probe mixture consists of Chromosome Enumeration Probe (CEP) CEP 3 SpectrumRed, CEP 7 SpectrumGreen, CEP 17 SpectrumAqua and Locus Specific Identifier (LSI 9p21) SpectrumGold.

Chromosome enumeration probes based on centromeric probes for several chromosomes are available from Genzyme Corp., Cambridge, Mass.

Kits for labeling DNA probes for use in FISH are available from Mirus Bio Corp., Madison, Wis. Labels include Cy3™, fluorescein, rhodamine and biotin.

FISH procedures and protocols are described, by way of nonlimiting example, in “Introduction to Fluorescence In Situ Hybridization: Principles and Clinical Applications” 1st edition, Andreef M and Pinkel D (eds.), Wiley-Liss, New York, N.Y. (1999).

An example of a procedure for conducting a FISH analysis on fetal cells is given in Mergenthaler et al. (J. Histochem. Cytochem., 53 (3): 319-322, 2005). Babochkina, T, et al. (Arch. Gynecol. Obstet. 2005 273(3):166-9) describe FISH analysis of fetal cells in maternal blood. In this study total nuclei were obtained by hypotonic treatment followed by Carnoy's fixation.

In certain embodiments a method of prenatal genetic diagnosis of aneuploidy is disclosed that employs optimized automated procedures such as may be incorporated in an automated fluorescence microscope system. First, a specimen of blood from a pregnant human female is obtained. In common embodiments the blood sample or a portion thereof is lysed and, as a further option, a fraction of the specimen that is enriched in cell nuclei may be isolated. Lysis may be induced by exposure to hypotonic conditions, or similar mild treatments. An enriched specimen, if so obtained, may be depleted of other cellular components, such as cell membranes, other cellular organelles, and cytoplasmic components. Next, at least a portion of the specimen containing cells from the blood, or containing lysed cells, or enriched nuclei, is deposited on a microscope slide. Then the deposited specimen is contacted with at least one FISH probe specific for a chromosome known to exhibit aneuploidy, under experimental conditions that promote hybridization of the probe to a target nucleic acid sequence comprised in the nuclei. In certain embodiments contact with a FISH probe and hybridization may be conducted prior to deposition of a sample on the slide. Additionally, in various embodiments the specimen or sample is contacted with a generic fluorescent chromosome or nucleic acid stain such as DAPI. In various additional embodiments the lysed specimen is subjected to conditions that fix components within the nuclei prior to placement on a slide; whereas in alternative embodiments the lysed specimen deposited on the slide is subjected to conditions that fix components within the nuclei. Next, a fluorescent microscopic image of the specimen with any fluorescent chromosome stain and any fluorescent probe bound is obtained using an automated fluorescence microscope system, such that the image represents a chromosome having a FISH probe hybridized to it. In common embodiments, the automated microscope operates to obtain chromosomal images by orienting the image to contain fluorescent images of chromosomes, if a generic chromosome stain such as DAPI has been used. The image may then be analyzed for aneuploidy. In various embodiments the analysis in conducted automatically using software resident in the automated microscope system. In additional embodiments more than one FISH probe specific for the potentially aneuploid chromosome may be used. The plurality of probes may include the identical fluorescent label, or different labels may be used in different probes. In various particular embodiments, the chromosome targeted by the FISH probe is chromosome 21, when it is intended to analyze for Down syndrome. In various other embodiments, the targeted chromosome may be chromosome 13, 18 or 16.

In additional embodiments a method for automated prenatal genetic diagnosis of aneuploidy is provided. In these embodiments, first, similar procedures for obtaining a specimen of blood from a pregnant human female, and similar various optional procedures for preparing, staining, hybridizing and depositing the specimen on a microscope slide at least one specimen on a microscope slide are carried out as have been described in the preceding paragraph. In various embodiments only one, or more than one, FISH probe specific for the potentially aneuploid chromosome may be used. The plurality of probes may include the identical fluorescent label, or different labels may be used in different probes. Next, at least one specimen-bearing slide is installed in a means for automated, reversible, placement of the slide on the stage of an automated fluorescence microscope. In various embodiments the means includes a slide-holding cassette, and the cassette may be controlled for automated operation. Next, the slide is caused to be reversibly placed on the microscope stage. This process is reversible such that a given slide may be withdrawn under automated operation, after an image is captured, and a new slide placed on the stage. Next, the microscope is caused automatically to obtain at least one image of the specimen including a locus on the slide comprising a fluorescent probe bound to a chromosome. In various embodiments automated operation of the microscope seeks out a plurality of loci within the specimen whose image contains a fluorescent probe bound to a chromosome. Next, the image is analyzed in an automated fashion in order to assess the state of ploidy of a chromosome at the locus. In various particular embodiments, the chromosome targeted by the FISH probe is chromosome 21, when it is intended to analyze for Down syndrome. In various other embodiments, the targeted chromosome may be chromosome 13, 18 or 16.

Computer and image processing technologies are constantly changing. Newer technologies which meet the needs of the above-described methods and apparatus, while not specifically described here, are clearly contemplated as within the invention. For example, certain conventional pixel and image file formats are mentioned above, but others may also be used. Image files may be compressed using JPEG or GIF techniques now known in the art or other techniques yet to be developed. Processing may be performed in an RGB color description space instead of the HLS space currently used. Other color spaces may also be used, as desired by the skilled artisan, particularly when detection of a sought-after characteristic is enhanced thereby.

While the embodiments of the invention have been described in connection with unenriched samples of maternal blood, aspects of the invention may be practiced on conventionally enriched or partially enriched maternal blood samples, as well. The use of a computer-controlled microscopic vision system to identify and to diagnose fetal cells within the sample is applicable to samples covering a full range of fetal cell concentrations. As has been discussed above, the use of such a system is particularly advantageous when used in connection with unenriched maternal blood samples.

The present invention has now been described in connection with a number of particular embodiments thereof. Additional variations should now be evident to those skilled in the art, and are contemplated as falling within the scope of the invention, which is limited only by the claims appended hereto and equivalents thereof.

### Statement Regarding Preferred Embodiments

While the invention has been described with respect to the foregoing, those skilled in the art will readily appreciate that various changes and/or modifications can be made to the invention without departing from the spirit or scope of the invention as defined by the appended claims.

What is claimed is:

1. A method of automated prenatal genetic diagnosis of aneuploidy comprising the steps of:
a) obtaining a specimen of blood from a pregnant human female;
b) exposing at least a portion of the specimen to conditions that lyse cells to provide cell nuclei;
c) depositing the lysed specimen on a microscope slide;
d) contacting the deposited lysed specimen with at least one in situ hybridization (FISH) probe specific for a chromosome known to exhibit aneuploidy under conditions that promote hybridization of the probe to a target nucleic acid sequence comprised in the nuclei;
e) using an automated fluorescence microscope to automatically obtain a fluorescent microscopic image of the contacted specimen comprising a chromosome having a FISH probe hybridized to it;
f) performing automated analysis of the image for a state of ploidy of the probed chromosome; and
g) automatically reporting results of the analysis;
wherein steps e)-g) are carried out without human intervention.

2. A method for high throughput prenatal genetic diagnosis of aneuploidy comprising the steps of:
a) providing at least one microscope slide comprising a specimen thereon, wherein the specimen comprises nuclei derived from a sample of blood from a pregnant human female that has been hybridized to at least one in situ hybridization (FISH) probe specific for a chromosome that may exhibit aneuploidy;
b) installing the at least one specimen-bearing slide in a means for automated, reversible, placement of the slide on the stage of an automated fluorescence microscope;
c) causing a specimen-bearing slide resident in the means to be reversibly placed on the microscope stage;
d) causing the microscope automatically to obtain at least one image of the specimen wherein the image comprises a representation of a FISH probe hybridized to a chromosome;
e) causing automated analysis of the image in order to assess the state of ploidy of the chromosome at the locus;
f) automatically reporting results of the analysis; and
g) repeating steps c)-f)
wherein steps c)-g) are carried out without human intervention.
